(19) United States
(12) Patent Application Publication
(10) Pub. No.: US 2004/0101901 A1
(43) Pub. Date: May 27, 2004

(54) Novel lipophilic complexes for insertion of receptors into lipid membranes

(76) Inventor: Piotr Tabaczewski, Richardson, TX (US)

Correspondence Address: BAKER & BOTTS, 30 ROCKEFELLER PLAZA, NEW YORK, NY, 10112

(21) Appl. No.: 10/305,333

(22) Filed: Nov. 27, 2002

(51) Int. Cl. G01N 33/53; C07K 14/705

(52) U.S. Cl. 435/7.1; 530/350; 514/395

(57) ABSTRACT

The present invention discloses novel compositions comprising a soluble lipid/receptor complex having a soluble receptor, attached in a site-specific manner to a water soluble lipid, which are amenable for insertion into lipid bilayers. The complexes of the invention are soluble in the absence of detergent. The present invention encompasses the compositions of the invention, methods of using the invention, and methods of producing the compositions of the invention.

### SPECIFICATION

The present invention relates to novel compositions comprising lipid/receptor complexes which comprise a water soluble lipid conjugated to a receptor, which are amenable for insertion into lipid membranes. These complexes have the property of being soluble in water in the absence of detergent. This invention relates to these compositions, their use as therapeutic agents, and methods of producing these compositions.

### BACKGROUND OF THE INVENTION

All biological membranes exhibit a bilayer of lipid molecules. Cells may comprise a plasma membrane and specialized internal membranes. The plasma membrane functions as a barrier between the cell and its environment, provides some structural support and regulates communication with the external environment. In animal cells, the plasma membrane contains the following classes of lipids—cholesterol, phospholipids (such as phosphatidylcholine, phosphatidylethanolamine, phosphatidylserine, and sphingomyelin), and glycolipids, such as cerebrosides. A phospholipid consists of a phosphorylated alcohol head group, a glycerol backbone, and two fatty acid sidechains. The fatty acid sidechains, found in both phospholipids and glycolipids, typically have between 14 and 24 carbons, usually 16 or 18 carbons.

The distribution of phospholipids in the plasma membrane creates a polarized structure (reviewed by Zwaal, Robert F. A., Schroit A. J., Pathophysiologic implications of membrane phospholipid asymmetry in blood cells. Blood, 1997, 89:1121-1132; Bevers, E. M., Comfurius P., Dekkers, D. W. C., Zwaal, R. F. A. Lipids translocation across the plasma membrane of mammalian cells. Biochimica et Biophysica Acta, 1999, 1349:317-330). Phosphatidylcholine and sphingomyelin are predominantly in the outer leaflet of the membrane and phosphatidylethanolamine and acidic phospholipids, such as phosphatidylserine, phosphatidylinositol, phosphatidic acid and their lyso-forms, are predominantly found on the inner, cytoplasmic leaflet of the plasma membrane.

The phospholipid gradient is maintained by the concerted action of energy driven enzymes, such as aminophospholipid translocase and flopase. Lipid scramblase, which promotes bi-directional movement of lipids across the bilayer regardless of head group, can mediate a rapid collapse of membrane asymmetry. This results in potentially reversible migration of significant amounts of acidic phospholipids to the outer layer of membrane (Hamill, A. K., Uhr, J. W., Scheuermann, R. H., Annexin V staining due to loss of membrane asymmetry can be reversible and precede commitment to apoptotic death, Experimental Cell Research., 1999, 251:16-21). Disappearance of phospholipid gradient with concomitant increase of cell surface acidic phospholipids has been described during environmental challenges including osmotic shock (Sims, P. J., Wiedmer T., Unraveling the mysteries of phospholipid scrambling, Thrombosis & Haemostasis, 2001 86:266-75; Rauch, C., Farge, E., Endocytosis switch controlled by transmembrane osmotic pressure and phospholipid asymmetry, Biophysical Journal, 2000, 78:3036-47), cell activation (Martin, S., Pombo, I., Poncet, P., David, B., Arock, M., Blank, U., Immunologic stimulation of mast cells leads to the reversible exposure of phosphatidylserine in the absence of apoptosis, International Archives of Allergy & Immunology, 2000, 123:249-58), tumorgenicity (Utsugi, T., Schroit, A. J., Connor, J., Bucana, C. D., Fidler, I. J., Elevated expression of phosphatidylserine in the outer membrane leaflet of human tumor cells and recognition by activated human blood monocytes, Cancer Res., 1991, 51:3062-3066) and physiological aging (de Jong, K., Beleznay, Z., Ott, P., Phospholipid asymmetry in red blood cells and spectrin-free vesicles during prolonged storage, Biochimica and Biophysica Acta, 1996, 1281:101-10). Increased concentration of acidic phospholipid on the outer membrane leaflet can be recognized by macrophages and lead to elimination by the immune system. (Fadok, V. A., de Cathelineau, A., Daleke, D. L., Henson, P. M., Braton, D. L., Loss of phospholipid asymmetry and surface exposure of phosphatidylserine is required for phagocytosis of apoptotic cells by macrophages and fibroblasts, J Biol. Chem., 2001, 276:1071-7).

The asymmetrical distribution of lipids in the plasma membrane also contributes to the overall charge of the membrane. While cholesterol and most membrane phospholipids do not possess net charge, acidic phospholipids have net negative charge. Since there are no net positively charged membrane lipids, plasma membrane lipids have an excess of negative charge in the neutral (pH 7.1-7.3) environment of body fluids.

A variety of proteins in the plasma membrane function to regulate the entry of materials into and exit of materials out of the cell, as well as communicate with the external environment of the cell. They may regulate such vital processes as differentiation, trafficking, cell death or survival, immunotolerance and the immune response. These proteins may be peripherally associated with the membrane or possess a segment that is embedded within the bilayer. The latter group, referred to as integral membrane proteins, may (1) be buried within the bilayer, (2) be attached by a lipid group to one face of the bilayer or (3) span the width of the bilayer. Membrane proteins are transcribed by the cell and delivered to the plasma membrane through various mechanisms.

Traditionally, cells may be manipulated in vitro to express proteins on the cell membrane by transfection techniques, by which genetic material (DNA or RNA) encoding the desired protein is delivered to the cell. These techniques include viral transduction using a defective or attenuated retroviral or other viral vector (see U.S. Pat. No. 4,980,286), or by direct injection or uptake of naked DNA, or by use of microparticle bombardment (e.g., a gene gun; Biolistic, Dupont), or coating with lipids or cell-surface receptors or transfecting agents, encapsulation in liposomes, microparticles, or microcapsules. These approaches, however, possesses numerous problems, including insufficient expression level, requirement for a dividing cell, requirement for selection drugs at the gene integration stage, poor performance relating to expression of particular proteins or number of proteins, limitations on synthesis based on cell type, etc.

An alternative approach to transfection relates to the direct protein transfer of the desired protein to the target cell membrane subsequent to the purification of the protein or synthesis of the protein in vitro. These approaches may encompass the use of cell or liposomal fusion. (Westerman and Jensen, Protein transfer of the costimulatory molecule, B7-2 (CD86), into tumor membrane liposomes as a novel cell-free vaccine, J. Immunol. Meth., 2000, 236:77-87.) A cell or liposome, expressing on its surface the desired protein, is fused with the target membrane to deliver the desired protein to the target membrane.

Lipophilic anchors may also be employed to transfer proteins to a target membrane. The lipid anchor may include palmitic acid, glycosylphosphatidylinositol (GPI) anchor, a hydrophobic region of the protein, and metal chelator lipids as discussed below.

N-hydroxysuccinimide ester of palmitic acid has been used to derivatize antibodies and protein A for transfer to cellular membranes (Colsky et al., Palmitate-derivatized antibodies can function as surrogate receptors for mediating specific cell-cell interactions, J Immunol Methods., 1989, 124:179-87; Kim et al. The use of palmitate-conjugated protein A for coating cells with artificial receptors which facilitate intercellular interactions, Journal of Immunological Methods, 1993, 158:57-65). Palmitic acid possesses a long hydrophobic chain of sixteen carbons. Phospholipids with long fatty acid sidechains having sixteen or eighteen carbons are the most common in biological membranes. These lipidated proteins were then transferred onto cell membranes.

Although this approach is relatively inexpensive and simple, can be applied to a number of cell types, and the proteins retain their function, the transfer of proteins does not provide expression at physiological levels. MHC antigens, for example, are expressed at several hundred thousand molecules/cell. Palmitate modification facilitated transfer of a maximum of 37,000 receptors on tumor cell A22.E10 without affecting cell viability during a prolonged incubation time of 60 minutes in the presence of a detergent (Kim et al., The use of palmitate-conjugated protein A for coating cells with artificial receptors which facilitate intercellular interactions Journal of Immunological Methods, 1993, 158:57-65). Although better receptor transfer efficiency, i.e. up to 165,000 receptors, has been reported using higher concentrations of derivatized receptor stock, the viability of the treated A22.E10 cells decreased to 78% (Kim et al. The use of palmitate-conjugated protein A for coating cells with artificial receptors which facilitate intercellular interactions, Journal of Immunological Methods, 1993, 158:57-65). This toxic effect was caused not by the lipidated receptor, but rather by increased amounts of detergent in the receptor transfer mixture. Thus, palmitate conjugation to receptor limits the number of transferred proteins to about 40,000/tumor cell. Since transfer efficiency is proportional to the cell surface area and murine lymphoma cells A22.E10 are rather large, it is expected that transfer capacity onto usually smaller non-tumor cells would be lower and would not approach physiological levels for many receptors.

Furthermore, derivatization is not particularly selective, since the N-hydroxysuccinimide ester of palmitic acid will attach to any accessible lysine residue.

A second approach utilized the glycosylphosphatidylinositol (GPI) anchor. The naturally occurring GPI anchor consists of a phospholipid tail, inositol, glucosamine, and three mannose residues with ethanolamine attached, and this anchor attaches to either the carboxyl- or amino-terminus of a protein receptor. Addition of a GPI signal sequence at either end of the receptor gene can yield receptor proteins capable of being modified with a GPI anchor (Cariappa, A., D. C. Flyer, C. T. Rollins, D. C. Roopenian, R. A. Flavell, D. Brown and G. L. Waneck., Glycosylphosphatidylinositol-anchored H-2D^{b} molecules are defective in antigen processing and presentation to cytotoxic T lymphocytes, European Journal of Immunology., 1996, 26:2215-2224; Brunschwig, E. B., E. Levine, U. Trefzer and M. L. Tykocinski., Glycosylphosphatidylinositol-modified murine B7-1 and B7-2 retain costimulator function, Journal of Immunology., 1995, 155:5498-5505; Poloso, N., S. Nagarajan, G. W. Bumgarner. J. C. Zampell and P. Selvaraj., Designer cancer vaccines made easy: protein transfer of immunostimulatory molecules for use in therapeutic tumor vaccines, Frontiers in Bioscience., 2001, 6:760-775). The GPI-anchored proteins can be expressed, purified, inserted into liposomes, and transferred onto cell membranes. A related and less expensive approach involves the insertion of a hydrophobic domain comprising non-polar hydrophobic amino acid residues to a protein to facilitate association with the hydrophobic core of a lipid bilayer (Wahlsten et al., Antitumor response elicited by a superantigen-transmembrane sequence fusion protein anchored onto tumor cells, J. of Immun., 1998, 161:6761-6767; U.S. Pat. No. 5,882,645). Both approaches allow for a site-specific insertion of a lipophilic anchor and yield functional protein receptors, but the method does not allow for the assembly of multicomponent protein complexes.

A third approach used metal chelator lipids (Dorn, I. T., Pawlitschko, K., Pettinger, S. C., Tampe, R., Orientation and two-dimensional organization of proteins at chelator lipid interfaces, Biological Chemistry, 1998, 379:1151-1159). Metal chelators such as iminodiacetic acid and nitrilotriacetic acid (NTA) can bind polyhistidine-tagged (His-tagged) recombinant proteins in the presence of Ni⁺⁺ or Zn⁺⁺. Metal chelators covalently bound to lipids can incorporate into cell membranes. This method is inexpensive, yields uniformly oriented and active receptors, and is applicable to proteins that have been modified with a genetically engineered His-tag. However, the link between the receptor and the membrane can be unstable in physiological conditions due to the presence of other divalent cations, lower pH, and reducing agents.

An inherent problem common to all the aforementioned lipid anchor protein transfer methods is the presence of detergent. Proteins derivatized with palmitic acid, a protein with having a GPI anchor, or a protein having a hydrophobic domain are not soluble in water. As a result, detergents must be present during storage, modification reactions, and transfer reactions. This characteristic limits these approaches to detergent-compatible receptors. The presence of detergent disrupts cell membranes and could pose a risk of toxicity in vivo. Furthermore, solubilizing agents, such as detergents, are hydrophobic and can be inadvertently incorporated into the membranes during the transfer reaction. Since the detergent is in large excess to the protein in the transfer mixture reactions, its presence can also limit the transfer capacity of the proteins.

Any method for the transfer of proteins onto a target bilayer, membrane or cell must meet a number of requirements in order to be useful. First, the method must allow for the transfer of at least a physiological amount of the desired protein to the target bilayer, membrane or cell. Second, the method must be applicable to a range of proteins. Third, the method must be applicable to a range of target bilayers, membranes or cells. Fourth, the method of the technique cannot disrupt protein function and orientation. Fifth, the transferred protein must remain on the target bilayer, membrane or cell for a sufficient amount of time. Sixth, the method should be inexpensive, reproducible, and simple to use. Seventh, the method should not functionally alter the target membrane. In other words, the method must not produce any cytotoxic or lipid-disruptive effects. There is a desire for a method of protein transfer that meets the above requirements.

The present invention overcomes the problems inherent in the earlier approaches for introducing proteins onto lipid membranes. In particular, the invention describes a method that does not involve the use of membrane-disruptive detergents to solubilize the protein prior to transfer of the protein to membranes.

### SUMMARY OF THE INVENTION

The present invention relates to novel composition comprising soluble lipid/receptor complex which comprise a water soluble lipid conjugated to a soluble receptor, which are amenable for insertion into lipid membranes in a detergent free manner. These complexes are amphipathic receptor/lipid complexes, which are highly soluble in aqueous media in the absence of detergents or other solubilizing agents and retain an affinity for lipid membranes.

The complexes of the invention comprise a soluble lipid and a soluble receptor. The receptor may be a protein, glycoprotein, polysaccharide or glycolipid. The receptor is preferably a protein.

In another aspect, the present invention provides for methods of producing the complexes of the invention.

Further, the present invention provides for an immunogenic composition capable of stimulating an immune response.

In an additional aspect, the present invention provides for methods of stimulating an immune response in an animal.

### BRIEF DESCRIPTION OF THE DRAWINGS

The following drawings form part of the present specification and are included to demonstrate further certain aspects of the present invention. The invention may be better understood by reference to one or more of these drawings in combination with the detailed description of the invention presented herein:

FIG. 1 shows a schematic of complexes of the invention inserted into a lipid membrane. FIG. 1A shows the complex comprising a phospholipid with a native charge distribution. FIG. 1B shows the complex comprising a lipid with a reversed charge distribution.

FIG. 2 shows a schematic of a complex of the invention.

FIG. 3 is a schematic diagram of the linkers that maybe used in the invention.

FIG. 4 is a schematic diagram of a bridging peptide and uses with a modified receptor.

FIG. 5 shows a FACS scan of murine RMA-S cells having avidin/lipid complexes transferred into the plasma membrane of the cells.

FIG. 6 shows a panel of FACS plots demonstrating the efficiency of proteins transferred into the plasma membrane of various cell types via a lipid protein xomplex of the invention—murine splenocytes (A and B), erythrocytes (C) and p815 tumor cells (D).

FIG. 7 is a graph showing cell viability and transfer efficiency of p815 tumor cells at various concentrations of avidin/lipid stock.

FIG. 8 is a graph showing the half life of transferred proteins on splenocytes, p815 cells and erythrocytes.

FIG. 9A shows the generalized antigen-independent response of splenocytes with transferred stimulatory antibodies anti-CD28 and anti-CD 3 antibodies transferred on its cell surface.

FIG. 9B is a bar graph showing the proliferation of B10P1 splenocytes with transferred proteins, CD28 and CD3 (A) and CD28 and I-A^{u}BIO (B).

FIG. 10 is a FACS analysis showing transfer of an avidin/lipid complex onto membranes of cells residing in its intact tissue environment in vivo.

### DETAILED DESCRIPTION OF THE INVENTION

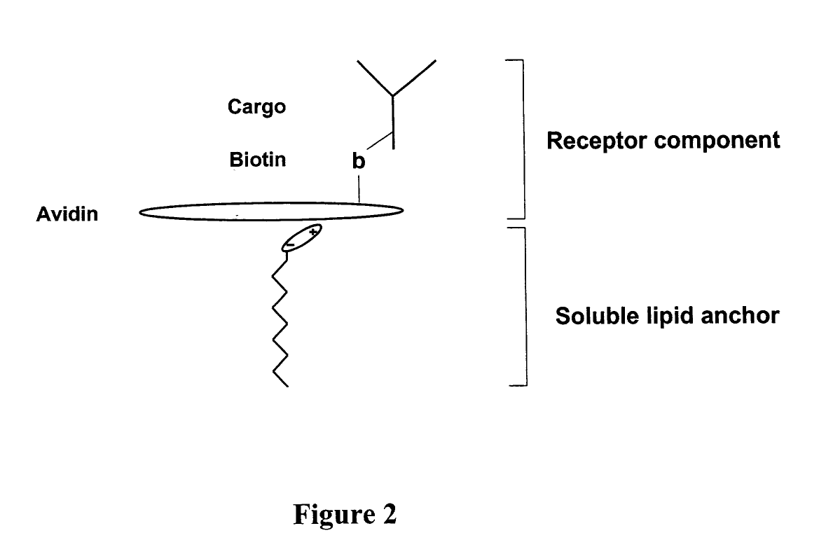

The present invention relates to a novel complex comprising a water-soluble receptor and a water soluble lipid, attached in a site-specific manner which is amenable for insertion into lipid membranes (FIG. 2). The receptor may be a protein, glycoprotein, polysaccharide, or glycolipid. The novel compositions comprise amphipathic receptor/lipid complexes, which are soluble in aqueous media in the absence of detergent or other solubilizing agents. The invention is based on the observation that proteins derivatized with short carbon chain soluble lipids remain soluble in aqueous media, while exhibiting a strong affinity for lipid bilayers. The present invention encompasses the compositions of the invention, methods of using the invention, and methods of synthesizing the compositions of the invention.

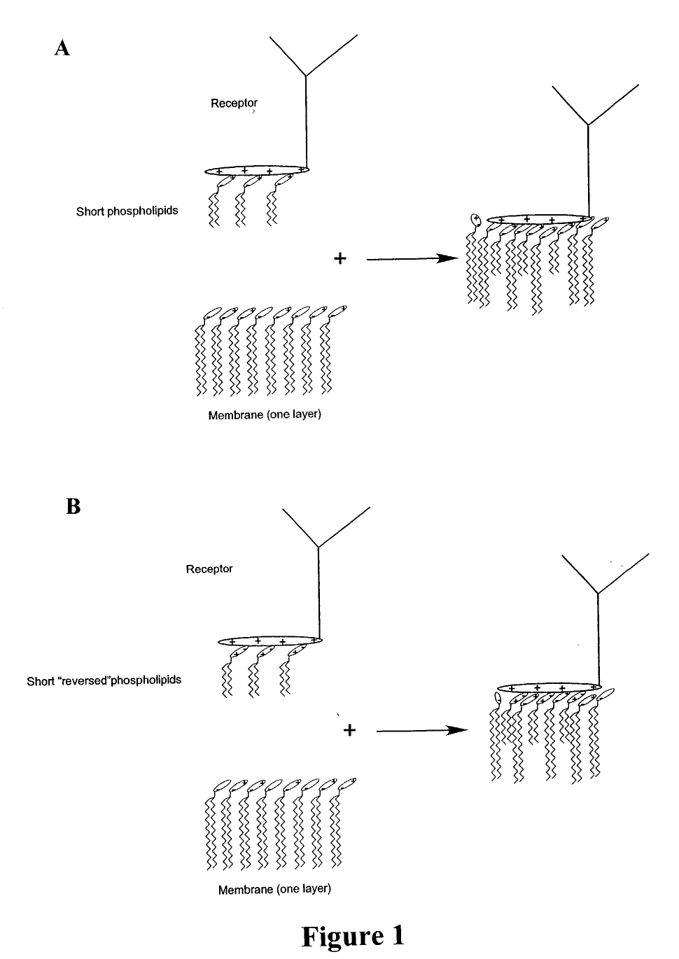

The compositions of the present invention share general characteristics of being soluble in water or aqueous media, in the absence of detergent or other solubilizers, while retaining some hydrophobic nature to allow for insertion of the complex into a target bilayer, membrane or cell. This ability is due, in part, to the presence of the short hydrophobic carbon chain fatty acid chains in the lipid (FIGS. 1 and 2). The hydrophobic nature of the fatty acid chain facilitates strong interaction with the hydrophobic core of a membranes. The short length of the sidechain also contributes to the solubility of the complex.

Furthermore, the compositions do not require the use of membrane-disrupting detergents or other solubilizing agents in their storage, assembly or transfer to a target lipid bilayer. The methods employed in the prior art for modifying proteins and inserting them into lipid bilayers does not escape the use of detergents. The disruptive characteristic of detergents can lead to toxic side effects and prevent the use of these methods in vivo. The elimination of the need for detergents in this invention is a significant step forward in the art.

In the present invention, the soluble lipid portion of the complex of the invention may comprise lipids that are water-soluble at relatively high concentrations. Preferably, the solubility of the lipids range from 20 μM to 1.2M. The solubility of the lipid contributes significantly to the solubility of the amphipathic receptor/lipid complex of the present invention.

Water soluble is defined herein as being retained in water solution after high-speed centrifugation, i.e. up to 105 000 g for 1 h, without the addition of solubilizing agents, such as detergents. For hydrophobic residues, maximal water solubility (saturating concentration) is the concentration equal to critical micellar unit (CMC) value (Hjelmeland, L. M and A. Chrambach., Methods in Enzymology., 1984, 104:305).

The soluble lipid portion of the complex of the invention is amphipathic, comprising a charged head and a hydrophobic tail.

In one embodiment of the invention, the lipid is a phospholipid. Water solubility of lipids in water is inversely correlated with the length of fatty acid side chains. Phospholipids with short fatty acid carbon sidechains are more soluble than phospholipids having greater than 12 carbons in their sidechains, such as those generally found in cellular membranes. The solubility of the phospholipid used in the lipid portion of the composition of the present invention is attributed to the length of the fatty acid side chain found on the phospholipid.

In a preferred embodiment of the invention, the phospholipids have two fatty acid sidechains that are each less than 12 carbons long. Preferably, the fatty acid sidechains are less than 9 carbons long. Having a shorter fatty acid sidechain confers a greater solubility to the composition.

In addition, short carbon chain phospholipids are particularly suited because the free amino group on the charged polar head can be used for conjugation of the lipid to the receptor to be derivatized, preferably those lipids based on phosphatidylathanolamine and phosphatidylserine. In addition, these lipids are structurally similar to, albeit shorter than the naturally occurring membrane phospholipid, phosphatidylethanolamine. Acceptable lipids include, but are not limited to, 8:0 1,2-dioctanoyl-SN-glycero-3-phosphoethanolamine, 6:0 N-NBD phosphatidylethanolamine, and 1,2-diacyl-SN-glycero-3-phosphoethanolamine.

In addition to phospholipids, fatty acids and lipids having a short, saturated or unsaturated single fatty acid chain from caproic to dodecanoic (from C6 to C12), or compound lipids having charged polar head groups, such as lysophospholipids, may also be suitable for use as the lipid portion of the compositions of the invention.

The present invention also contemplates the use of structurally variant clusters of phospholipids. It is anticipated that previously defined hydrophobic building blocks of variable length of carbon chain will be combined in form of irregular clusters. This type of structure will have higher affinity to planar membranes rather than for itself.

Preferably, the soluble lipid of the present invention comprises charged headgroups. The phosphorylated alcohol head groups found on phospholipids in cellular membrane comprise two charged components, a negatively charged phosphate group and a positively charged alcohol (see FIG. 1A).

Alternatively, a water soluble short carbon chain lipid with a reverse charge head group can be used as the lipid in the composition of the invention (FIG. 1B). To avoid confusion with phospholipids, phospholipid-like components with reverse charge distribution of the head group will be called “cationic lipids.” Similar to the phospholipid described above, cationic lipids comprise a short carbon fatty acid sidechain, a charged head group and is soluble in water. Unlike a phospholipid molecule, the head group of a cationic lipid is positively charged. These lipids can be cationic quaternary ammonium salt lipids or lipoamines. Examples of such lipids include, but are not limited to, DMRIE (Dimyristoyl Rosenthal inhibitor ether), DOGS, DC-CHOI, DOPA, DLRIE, DOPE (Dioleoylphosphoethanolamine), etc. (Gerardo Byk et al. Journal of Medicinal Chemistry, 1998, 41:224-235). Many of the aforementioned cationic lipids are components of liposome-mediated transfection reagents, used in DNA transfection. The use of cationic lipids with positively charged headgroups may promote the initial electrostatic interaction of the complexes of the present invention to membranes.

Electrostatic interactions between the compositions of the invention and the outer layer of lipid membranes can facilitate initial transfer of the complexes of the invention onto the membranes. The inner leaflet of cytoplasmic membranes comprises approximately 30% acidic phospholipids. Various intercellular proteins interact with the inner layer of biological membranes via electrostatic interactions. They include cytochrome C, myelin basic protein and spectrin (Johnson et al., Coupling of spectrin and polylysine to phospholipid monolayers: Studied by specular reflection of neutrons, Biophys. J., 1991, 60:1017-1025). However, the outer leaflet of the plasma membrane may also comprise acidic phospholipids of variable concentrations and may be relied upon to facilitate electrostatic interactions. Positively charged molecules have been shown to adhere efficiently to cell membranes (Kobayashi Y., Onuki H., Tachibana K., Mechanism of hemolysis and erythrocyte transformation caused by lipogrammistin-A, a lipophilic and acylated cyclic polyamine from the skin secretion of soapfishes (Grammistidae)., Bioorganic & Medicinal Chemistry., 1999, 7:2073-2081; Mayhew E., Harlos J. P., Juliano R. L., The effect of polycations on cell membrane stability and transport process, J. Membrane Biol., 1973, 14:213-228). In addition, the positive charge of avidin has been identified as the reason for unspecific cell binding and the source of the background in cell staining reactions. To eliminate this interaction, avidin prepared without a net charge (NeutrAvidin™, Pierce, Rockford, Ill.) is commercially available. Furthermore, it is well known in the art that coating a substrate with a basic amino acid, such as poly L-lysine promotes cultured cell attachment via electrostatic interactions.

To promote electrostatic interactions between the outer membrane and the compositions of the present invention, the target membrane may be provided with a higher proportion of acidic phospholipids. This may be achieved by preincubation of the target membrane with acidic phospholipids generate a more negatively charged outer membrane surface.

In some embodiments of the invention, the soluble lipid of the composition can be modified. The modification may comprise the addition of a label to estimate the efficiency of the transfer to a target membrane and to evaluate membrane distribution. The modification may entail the addition of a fluorescent or enzymatic tag. For example, if 6:0-N-NBD phosphatidylethanolamine is used, the phospholipid can be labeled with a fluorochrome for detection in flow cytometry at an excitation wavelength of 460 nm and an emission wavelength of 534 nm. Flow cytometry can be extended to sort cells exhibiting the fluorescent label from cells that do not, thereby separating cells having the composition bound to their surfaces from those that do not. Tracking movement of the labeled surface transferred protein also may allow for studying the interaction between receptors found on two interacting cells.

The receptor component of the receptor/lipid complex of the invention can be a protein glycoprotein, polysaccharide or glycolipid. In a preferred embodiment of the invention, the receptor is a glycoprotein, such as avidin, which is utilized in a non-limiting fashion to exemplify the present invention.

The means of attaching the lipid to the receptor can be any interaction that is site-specific and does not adversely affect the functional activity of the receptor. It can be a covalent or non-covalent attachment. Any coupling reaction to the receptor that allows for site-specific addition of short carbon chain, water-soluble lipids is appropriate. For example, additional cysteines can be introduced into the receptor via known genetic engineering protocols to allow for coupling via disulfide bridges. Reactive head groups on phospholipids, such as the amine group of phosphatidylethanolamine, can be covalently linked to thiol groups found on cysteine residues of a protein. Various commercial crosslinking reagents are available that may be used to attach the lipid to the receptor. Other reactions include the use of compound lipids having head groups containing iodoacetate and related α-haloketo compounds as bromoalkanoic acids, chloroalkanoic acids and related amides or N′-ethylmaleimide and its derivatives or bimane derivatives or disulfide reagents as 5,5′-dithiobis-(2-nitrobenzoic acid), 2,4-dinitrophenyl-cysteinyl disulfide and related compounds.

As stated above, a preferred receptor is a glycoprotein. Attaching the glycoprotein to the soluble lipid through a mannose residue in a glycosyl unit of the glycoprotein is particularly advantageous. Glycoproteins have a limited and well-defined number of glycosylation sites, allowing for controlled site-specific conjugation. There are a number of mannose residues which provide a variable number of conjugation sites. Furthermore, many glycosylation sites are not involved in the physiological function of the glycoprotein. Thus, glycosylation sites can be blocked by conjugation to a lipid without any effect on glycoprotein function. In the absence of a glycoprotein, the sequence for the receptor of interest may be manipulated to insert glycosylation signals at desired locations to generate a glycoprotein.

Avidin, a 66 kDa glycoprotein found in egg whites, is a commercially available, highly water-soluble (up to 20 mg/ml), extremely stable and relatively non-expensive, basic (isoelectric point=10.5) tetrameric glycoprotein (Green, N. M., Avidin, Biochemical Journal., 1963, 89:585-620; Pugliese, L., A. Coda, M. Malcovati and M. Bolognesi., Three-dimensional structure of tetragonal crystal form of egg-white avidin and its functional complex with biotin at 2.7 A resolution, Journal Molecular Biology, 1993, 231:698-710). Its molecular size of 66 kDa is representative of the average size of an integral membrane protein receptor.

Glycosylated mannose residues on the avidin molecule can be modified to act as acceptor sites for lipid conjugation. Each of the four avidin subunits contains a single glycosylation site, at residue Asn 17 (Bruch, C. R. and H. B. White., Compositional and Structural Heterogeneity of Avidin Glycopeptides, Biochemistry, 1982, 21:5334-5341). The oligosaccharide chains on avidin each contain approximately four to five mannose residues. Sodium periodate can be used to oxidize mannose residues present in the oligosaccharide chains, which results in the selective oxidation of vicinal diols. The oxidized mannose can be conjugated to phosphatidylethanolamine derivatives to produce an imine linkage, which is then reduced by sodium cyanoborohydride to yield the more stable amino linkage. This reaction is simple to conduct, reproducible, and proceeds in mild conditions.

Sodium periodate will also selectively oxidize N-terminal serine and threonine residues to aldehyde groups to which phosphatidylethanolamine can be coupled. Alternatively, aldehydes can be introduced in glycosyl units using galactose oxidase. In addition, water soluble compound lipids having aldehyde or ketone reactivity containing hydrazine derivatives, e.g. hydrazide, semicarbazide, carbohydrazide, and similar compounds, in the head groups can be used instead of phosphatidylethanolamine.

Attachment of the soluble receptor and soluble lipid to each other may also be mediated by a solid substrate. In particular, lipid anchor units can be attached onto a solid support, such as beads. The receptor can be added to conjugated to reactive groups found on the lipids under receptor compatible conditions. After the reaction, the entire receptor/lipid complex can be cleaved from the solid support, chemically, enzymatically or with light photocleavable).

The target lipid membrane can be a bilayer or a monolayer. It may be the cell membrane of a viable cell, an isolated membrane, a lipid bilayer, or a lipid vesicle. Cell membranes can include the membranes of resting and activated splenocytes, erythrocytes, tumor cells, macrophages, dendritic cells, stem cells, muscle cells, nerve cells, etc.

Several methods can be employed to facilitate transfer of the receptor to the membrane. Transfer of the complexes should occur under physiological conditions (pH 7.0) to allow for the practice of the invention in vivo. In certain embodiments of the invention, transfer occurs in a buffer with low osmotic pressure and in the presence of bivalent cations to cause transiet collapse of membrane asymmetry. Additionally, the low ionic concentrations will not shield the charges on the lipid membranes and transferred receptor units.

Transfer of proteins may include intermediate steps. These intermediate steps include, inter alia, the use of carriers such as vesicles or beads for insertion into membranes. For example, soluble receptor/lipid complexes of the invention may be conjugated to a solid support, such as a bead, and mixed with target cells or membranes for the transfer of the complex to the membrane. This approach would be amenable for less water-soluble complexes that have high affinity for the membranes. The solid support may act as virtual detergent to prevent aggregation of the complex. The cleavage reaction of the complex from the solid substrate may occur in the presence of the cell to facilitate transfer and prevent self-aggregation.

In a preferred embodiment of the invention, the receptor component of the receptor/lipid complex may further comprise cargo. The cargo may be a a protein, glycoprotein, polysaccharide, or glycolipid. Preferably, the cargo is a protein or glycoprotein.

In an embodiment of the invention, desired cargo may be transferred to a lipid bilayer through a biotin-avidin mediated interaction. The cargo may be modified by conjugation to biotin molecules, which confers the capability to interact with avidin molecules. Since many proteins may be biotinylated, the use of avidin as the receptor is particularly preferred. It allows for the insertion of many types of proteins into lipid membranes.

Avidin serves as a receptor of the vitamin, biotin, and binds up to four molecules with a K_{d} of approximately 10⁻¹⁵ M. This strong and specific avidin-biotin interaction is the reason that avidin is one of the most useful and widely used reagents to in biology and medicine. Both avidin and biotin have been used extensively as “labels” for antibodies, fluorescent dyes, proteins and other molecules of interests.

Similar to the charged lipid component, electrostatic interactions between the receptor component of the receptor/lipid complex may also promote insertion of the complex into lipid membranes. Positively charged avidin molecules are attracted to the negatively charged membrane.

Alternatively, one can utilize the interaction between protein A or protein G proteins and the IgG instead of the avidin-biotin mediated interaction, to attach the cargo component of the receptor/lipid complex.

The cargo of the receptor component of the receptor/lipid complex of the present invention may be a peptide, polypeptide or protein (FIG. 2). Preferably, the cargo is a protein. In an embodiment of the invention, the protein is a transmembrane protein. To prepare the protein for site specific conjugation to a desired lipid to form the complex of the invention, the DNA sequence encoding the protein may be modified by the removal of leader sequences, and sequences encoding the cytoplasmic and transmembrane region. The sequences can be inserted into an expression vector for expression in bacterial or yeast systems that are well known to those in the art. The expressed protein can then be isolated from the cells and folded in vitro. Alternatively, the proteins can be obtained by in vitro transcription and translation using techniques known to those in the art.

Additionally, the sequence of the desired protein can be manipulated to obtain desired properties, such as addition of reactive sites for glycosylation, addition of reactive groups for the attachment to the lipid to the receptor, improved solubility, and increased membrane affinity. For example, the addition of cysteine residues in the protein of interest can be used to promote the reaction between the —SH group of a cysteine residue and 8:0 1,2-dioctanoyl-SN-glycero-3-aminoiodoacetamide (attachment to the lipid portion). Furthermore, a charged adapter sequence, such as a polylysine string, can also be added to promote membrane affinity.

Preferably, the protein is an immunogen or antigen. In an embodiment of the invention, the protein is B7.1 Other proteins include B7.2 and CD40L.

Elimination of tumor cells can occur through the action of cytotoxic cells, such as natural killer (NK) cells and cytolytic cells (CTLs). To initiate a T cell-mediated response, it has been shown that antigen processing cells (APC) present at least two signals to T cells. One signal involves presentation of the antigen by MHC molecules on APC to the T cell receptor (TCR) of T cells. A second signal delivered by the APC to the T cells involves a costimulatory molecule, such as B7.1, recognized by CD28 on the T cell. This additional signal is required for clonal expansion of T cells. B7.1 is normally expressed only on antigen presenting cells, macrophages and a subset of activated B cells. Tumor cells lacking B7 fail to deliver the costimulatory signal, resulting in a deficient immune response against tumor antigens. Expression of costimulatory proteins, such as B7.1 on tumor cells, on tumor cells and subjecting these cells to an individual can be used to improve tumor cell immunogenicity. Thus, a further embodiment of the invention is a receptor/lipid complex inserted into membranes for use as a synthetic vaccine, inorder to reduce, inter alia, antitumor cell immunity.

To optimize the use of B7.1 inserted into a membrane using a lipid/receptor complex of the invention as a synthetic vaccine, the wild type sequence can be modified. The leader sequence, sequences encoding the transmembrane and cytoplasmic regions can be deleted. Cysteine residues can be inserted into the B7.1 sequence to facilitate attachment to the lipid portion. Furthermore, an adapter sequence can be added to B7.1 gene to generate a charged stretch of polylysines at the C-terminal end of the translated B7.1 protein. The sequence is inserted into a yeast expression vector, such as pPIC3.5 (Invitrogen, Carlsbad, Calif.). The transformants can be induced to produce the B7.1 protein. Inclusion bodies from the yeast can be collected and B7.1 can be isolated. The protein can be folded in vitro.

In a further embodiment of the invention, the cargo component of the receptor/lipid complex comprises multiple peptides, polypeptides or proteins.

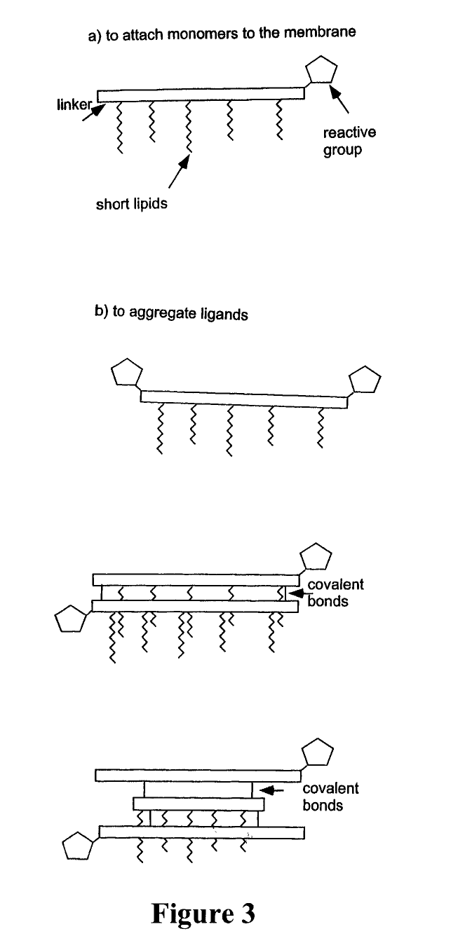

In yet another embodiment of the invention, the receptor/lipid complex further comprises a linker. FIG. 3 shows a schematic of this embodiment of the invention. The linker is an amphipathic, water soluble, lipophilic molecule comprising a reactive group for attachment of the linker to the receptor. The linker functions to attach the receptor component with the lipid component of the receptor/lipid complex. Linker molecules include, but are not limited to, polyamines, polyamino acids, oligosaccharides, polysaccharides, polyglycols, and oligonucleotides, including oligonucloetide variants such as as modified uridines.

Reactive groups on the linker molecules are used for attachment to the receptor component of the complexes of the invention. Reactive groups for sulthydryl groups include, but are not limited to, cysteine iodoacetate and related α-haloketo compounds, such as bromoalkanoic acids, chloroalkanoic acids and related amides, N′-ethylmaleimide and its derivatives, bimane derivatives, and disulfide reagents, such as 5,5′-dithiobis-(2-nitrobenzoic acid), 2,4-dinitrophenyl-cysteinyl disulfide and related compounds. Reactive groups for aldehyde groups of modified glycosyl units include primary amines, hydrazine derivatives (hydrazide, semicarbazide, carbohydrazide and similar compounds). These reactive groups are attached to one or both ends of the linker molecules for site-specific conjugation of the receptor.

The linker may be of variable length, preferably between 10 and 500 angstroms in length. The length of the linker is dependent upon the desired conformation of the resulting receptor/lipid complex. Distance of various proteins in a multi-protein receptor/lipid complex, for example, will vary based on the size of the protein and the desired rigidity of the complex, as discussed below.

The linker provides flexibility by allowing for the addition of various cargo to complexes of the invention. More linkers can be combined to provide desired lattice framework that controls spatial relationship and stoichiometry between receptors and serves as a membrane anchor unit. The linkers may be homobifunctional or heterobifunctional linkers, wherein the heterobifunctional linkers have different reactive groups to allow for conjugation of different cargo. More than one linker may be attached together by covalent bonds via sidechains to build crosslinked heteroduplex and heterotriplex complexes. The sidechains may be derivatized with a variety of reactive groups to allow for crosslinking reactions. In the case of modified oligonucleotides, linkers can be synthesized in the form of self-assembling structures, allowing for building multi-receptor signaling surfaces. The use of multiple linkers may also allow for control over the rigidity of the receptor/lipid complex.

The overall conformation of the receptor/lipid complex can be used advantageously to prevent formation of unwanted aggregates between complexes. External charged linkers also have the advantage of increasing solubility of the overall receptor/lipid complex by shielding the internal hydrophobic central region to increase membrane affinity.

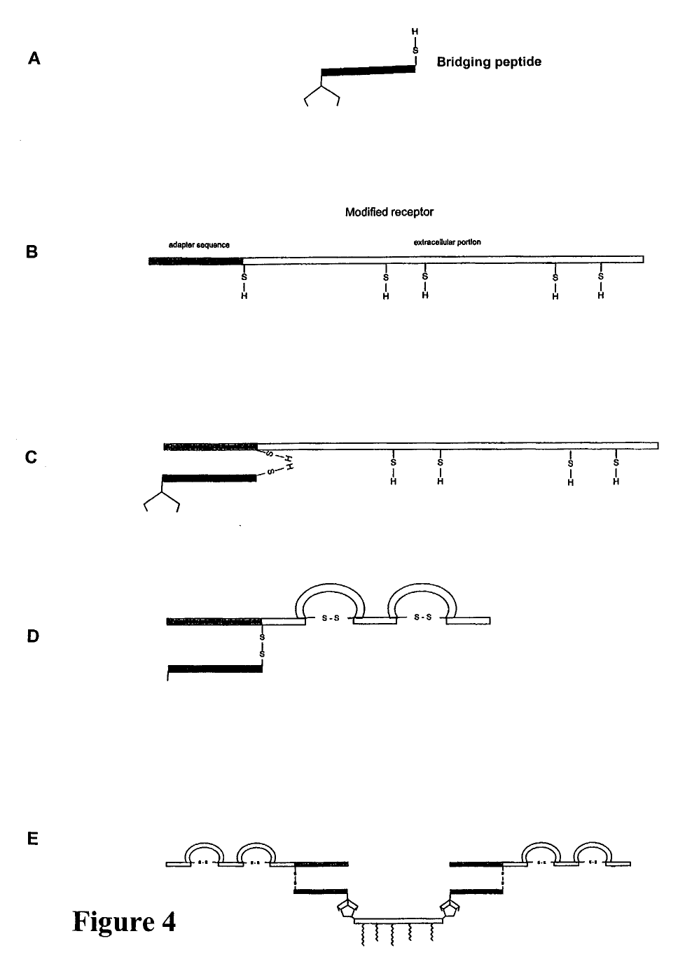

In an alternative embodiment of the invention, the protein may be a bridging peptide. As shown in FIG. 4A, the bridging peptide can be a short acidic peptide, comprising glutamic acid and aspartic acid, with a cysteine residue at one end of the peptide and a reactive group at the other end of the peptide. This bridging peptide can be used as means for attaching an additional desired protein to be transferred onto a membrane. The additional desired protein may comprise adapter sequence and cysteine residues (FIG. 4B). It is prepared and maintained in denatured form using 8M urea and 1 mM DTT. The additional desired protein can be incubated with the bridging peptide in a reducing environment in a 1:1 molar ratio to allow for electrostatically driven adhesion (FIG. 4C). It can then be folded, i.e. disulfide bond formation, purified, and reacted with an appropriate lipid in preparation for insertion or transfer onto membranes. Modification of this technology can also allow for the attachment of the bridging peptide to a folded and gently reduced receptor. In addition, the length of the bridging peptide may be variable based upon the size of the proteins to be transferred and the desired clustering of multiple proteins of the receptor/lipid complex. The use of a bridging peptide allows for greater flexibility in the designing multi-protein receptor/lipid complexes for transfer onto membranes.

In another embodiment, the present invention provides for methods of using the complexes of the invention for insertion of receptors into lipid bilayers. In an embodiment of the invention, complexes may be inserted into cells or tissues of a mammal. In a preferred embodiment, the mammal is human.

Included as an embodiment of the invention is a method of stimulating a protective immune response in an animal comprising introducing a novel complex of the present invention to a subject. The complex comprises an immunogenic or antigenic protein. In a preferred embodiment, the immunogen or antigen is biotinylated. The complex can be inserted into a membrane of a cells which are then introduced to a subject thereby to stimulate an immune system. According to the invention, synthetic vaccines may be prepared in this manner.

The Examples demonstrate that the soluble lipid/receptor complexes of the invention may be transferred successfully to cell membranes both in vitro and in vivo. Proteins have been transferred to membrane at physiological levels for over 4 days and to the membranes of multiple cell types including tumor cell lines. As a result, the present invention contemplates methods of using such complexes to stimulate an immune response.

In an embodiment of the invention, the soluble receptor/lipid complexes are inserted at physiologically relevant levels. In a further embodiment, 106 receptor/lipid complexes are inserted, e.g. 0.1 μg of a 60 kDa protein can be inserted into 10⁶ cells.

The present invention provides for physiological compositions comprising the soluble lipid/receptor complexes of the present invention. Aqueous physiological compositions of the present invention comprise an effective amount of a complex of the present invention or a physiologically or pharmaceutically acceptable salt thereof, dissolved and/or dispersed in a physiologically or pharmaceutically acceptable carrier and/or aqueous medium.

The phrases “physiologically, pharmaceutically and/or pharmacologically acceptable” refer to molecular entities and/or compositions that do not produce an adverse, allergic, and/or other untoward reaction when administered to an animal.

As used herein, “physiologically and/or pharmaceutically acceptable carrier” includes any and/or all solvents, dispersion media, coatings, antibacterial and/or antifungal agents, isotonic and/or absorption delaying agents, and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For human administration, preparations should meet sterility, pyrogenicity, general safety, and/or purity standards as required by FDA Office Biologics standards.

The methods of transfer can be applied to humans for the treatment of disease, such as cancer, HIV, CMV, and other therapeutic uses.

In an embodiment of the invention, the soluble lipid/receptor complexes may be inserted into cell or tissues for research purposes. Proteins associated with various disorders may be fluorescently tagged, inserted into cells and evaluated in imaging technologies. Tracking movement of the labeled surface transferred protein may allow for studying the interaction between receptors found on two interacting cells. When different proteins are transferred, each labeled with different fluorochromes, such as FITC, PE, APC, complex cellular interactions can be mapped. For this reason, it is anticipated that described technology and compositions will find significant applications in basic research.

The practice of the present invention employs, unless otherwise indicated, conventional techniques of synthetic organic chemistry, protein chemistry, molecular biology, microbiology, and recombinant DNA technology, which are well within the skill of those in the art.

### EXAMPLES

The following examples are included to demonstrate preferred embodiments of the invention. It should be appreciated by those of skill in the art that the techniques disclosed in the examples that follow represent techniques discovered that the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

### Example 1

### Transfer of Avidin Conjugated to Lipids with Short Carbon Fatty Acid Chains onto Cell Membranes of Murine RMA-S cells

Materials and Methods

Sodium periodate was used to oxidize mannose residues present in the carbohydrate units of glycosylation structures as acceptor sites in the avidin molecule. Avidin (5 mg/ml) was incubated with 4.5 mM sodium periodate in 6 mM EDTA, 55 mM acetate buffer (pH 6.0) for 60 minutes at room temperature. This reaction results in selective oxidation of vicinal diols of mannose by sodium periodate. The reaction was stopped by the addition of 3% v/v ethylene glycol and buffer was changed to 20 mM phosphate buffer (pH 7.2) using gel filtration on Sephadex G-25 column (Pharmacia, Peapack, N.J.)

Phospholipids with short (6-8) carbon fatty acid chains were incubated with the modified avidin molecules. 6:0-N-NBD phosphatidylethanolamine and 1,2-dioctanoyl-SN-glycero-3-phosphoethanolamine were used. Phospholipid with long chain fatty acid chain, i.e. 1,2-dioleoyl-SN-glycero-3-phosphoethanolamine (18 carbons) was also used as a control. Lipids are stored at 10% DMSO. Lipid was added at 40:1 (lipid:protein) molar ratio to the modified avidin and incubated for twelve hours at 4° C. The unreacted lipid was dialyzed away and the buffer was changed to 20 mM phosphate buffer (pH 6.5). NaBH₃CN (0.1 mg/ml) was added to the dialyzed mixture and incubated at room temperature for 3 hours. This reaction leads to reduction of hydrazones and increased stability of the avidin-lipid linkage. The reaction mixture was further dialyzed to remove unreacted reagents. The avidin derivatized stock was resuspended to 1.5 mg/ml in 20 mM phosphate buffer and stored at 4° C.

Murine tumor cells, RMA-S, was used in the experiment described in this Example. Tumor cells were grown to logarithmic phase and washed three times in PBS.

0.6 ml of avidin-derivatized lipid stock (1.5 mg/ml) was added to 2×10⁷/ml cells. The cells were incubated for 7 minutes at room temperature. To stop the reaction, equal volume of fetal calf serum was added. The cells were washed three times in PBS. Cells were stained with FITC biotin. The cells were subjected to fluorescent activated cell sorting (FACS) to detect the transfer of avidin to cells.

Results

Short, water-soluble phospholipids, 6:0-N-NBD phosphatidylethanolamine and 1,2-dioctanoyl-SN-glycero-3-phosphoethanolamine, were covalently attached to water-soluble glycoprotein avidin in a site-specific manner. These phospholipids have 6 and 8, respectively, carbon fatty acid chains. As a control, a long, water-insoluble phospholipids, 1,2-dioleoyl-SN-glycero-3-phosphoethanolamine, having a 18 carbon fatty acid chain was also used. Lipids of this length attached to sugar moieties resemble naturally occurring GPI anchors. Avidin derivatized with long lipids is structurally similar to GPI attached or engineered amphipathic receptors described and were used as a control reagent.

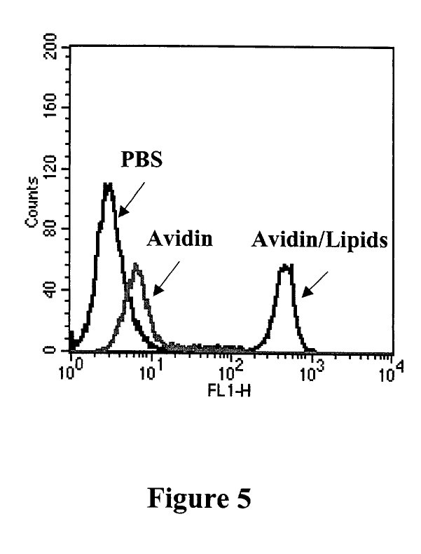

FIG. 5 shows the presence of transferred avidin molecules on the surface of cultured murine RMA-S cells. Cultured RMA-S cells incubated with 6:0-N-NBD phosphatidylethanolamine modified avidin stain positively with FITC-BIO reagent as compared with PBS-treated cells or cells incubated with unmodified, wild type avidin. This data shows that transfer of avidin on the cell surface of RMA-S cells is dependent upon lipid modification of avidin. Residual, low capacity binding of wild type avidin to the cell surface is caused by low affinity carbohydrate-glycocalix interactions and electrostatic avidin-membrane interactions.

### Example 2

### Transfer of Protein Complexes mediated by Avidin Conjugated to Lipids with Short Carbon Fatty Acid Chains onto Cell Membranes of Multiple Cell Types

Materials and Methods

Erythrocytes were obtained from mouse tail bleedings and collected into heparinized vials and processed using standard protocols. Fresh splenocytes were also obtained from mice. Both cell types were purified with discontinuous Ficoll gradient and washed three times in cold PBS. Splenocytes were isolated on the day of use. Erythrocytes could be stored for few days in PBS at 4° C.

To obtain activated splenocytes, freshly isolated splenocytes were resuspended at 5×10⁶/ml and cultured for three days in humidified incubator at 37° C. and 5% CO₂ in RPMI 1640 medium supplemented with 5 μg/ml of Concanavalin A (Con A), P-mercaptoethanol at 10⁻⁵M, 2 mM L-glutamine and 10% FCS.

Lipids from Example 1 were also used for these experiments.

0.6 ml of avidin-derivatized lipid stock (1.5 mg/ml) was added to 0.4 ml suspension of cell suspensions. The following cell preparations were used: 2×⁷/ml activated splenocytes, 5×10⁷/ml resting splenocytes, 2×10⁷/ml p815 cells and 5×10⁸/ml erythrocytes. The cells are incubated for 7 minutes at room temperature. To stop the reaction, equal volume of fetal calf serum was added. The cells were washed three times in PBS.

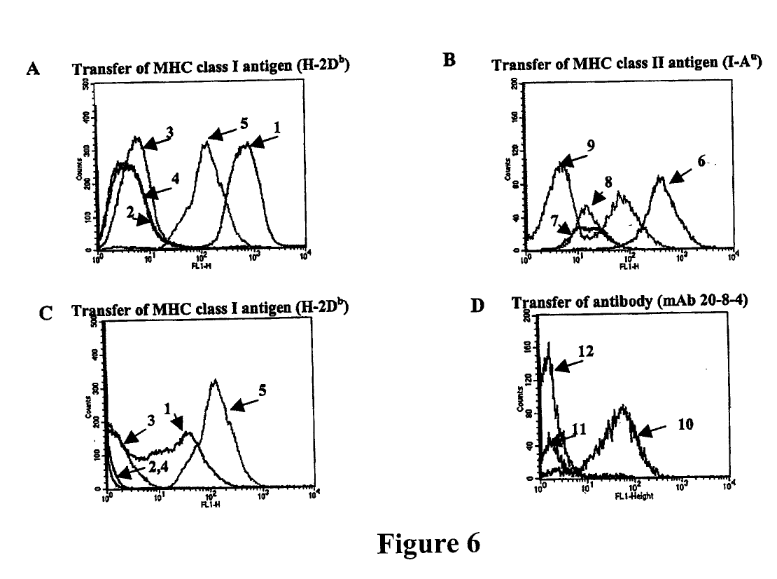

Avidin/lipid treated cells were incubated with biotinylated proteins to evaluate the efficiency of transfer of specific cargo to cell membranes (FIG. 6). Cells were incubated for 30 minutes on ice with 1001 μl of biotinylated protein receptors, resuspended at 10 μg/ml in 1% v/v dilution of FCS in PBS. The proteins include MHC class I antigens, H-2 D^{b} and H-2 D^{b}, MHC class II antigen, I-A^{u}, and monoclonal antibody, 20-8-4, which recognizes murine MHC antigens H-2K^{b}and Qa-2. Unbound reagents were removed by washing three times in ice cold PBS. The cells were incubated with antibodies to the proteins and prepared for flow cytometry. The following is a list of antibodies used and their corresponding antigens: mAb 28-14-8/FITC detects anti H-2D^{b}; anti I-A^{u}/FITC detects I-Au; goat anti mouse IgG/FITC detects mouse IgG. mAb 46/FITC is used to as a negative control to detect Qa-2. mAb 28-14-8/FITC and mAb46/FITC were prepared in the lab by the inventor. Goat anti-mouse IgG/FITC (ICN/Cappel, Aurora, Ohio), anti I-A^{u}/FITC (Pharmingen, San Diego, Calif.)

The scheme for various incubations are shown in Table 2 below.

TABLE 2
Various Incubation protocols for transfer of Avidin/Lipid
Complexes to Various Cell
Membranes (Panels refer to FIG. 6.)

| | | | |
|---|---|---|---|
| Inhibitor | Transfer Vehicle | Receptor | Antibody |

Activated Splenocytes and Erythrocytes (Panels A and C)

| | | | | |
|---|---|---|---|---|
| (1) | — | avidin/lipids | H-2D^{b}BIO | anti H-2D^{b} |
| (2) | biotin | avidin/lipids | H-2D^{b}BIO | anti H-2D^{b} |
| (3) | — | avidin/lipids | H-2D^{b}BIO | anti Qa-2 |
| (4) | — | — | H-2D^{b}BIO | anti H-2D^{b} |
| (5) | — | — | — | anti H-2D^{b} |

Activated Splenocytes (Panel B)

| | | | | |
|---|---|---|---|---|
| (6) | — | avidin/lipids | I-A^{u}BIO | anti I-A^{u} |
| (7) | biotin | avidin/lipids | I-A^{u}BIO | anti I-A^{u} |
| (8) | — | — | I-A^{u}BIO | anti I-A^{u} |
| (9) | — | — | — | anti I-A^{u} |

p815 Cells (Panel D)

| | | | | |
|---|---|---|---|---|
| (10) | — | avidin/lipids | mAb 20-8-4BIO | anti mouse IgG |
| (11) | — | avidin/lipids | — | anti mouse IgG |
| (12) | — | — | mAb 20-8-4BIO | anti mouse IgG |

Results

Normal murine splenocytes activated with Con A (FIG. 6, panels A and B), erythrocytes (FIG. 6, panel C) and p815 tumor cells (FIG. 6, panel D) were incubated with avidin/lipids and control reagents as indicated in Table 2. Cells were washed three times in PBS using centrifugation between individual incubation steps. The avidin/lipids driven transfer of biotinylated molecules was monitored in flow cytometry assay with fluorochrome labeled reagents recognizing transferred receptors as follows: anti H-2D^{b}-mAb 28-14-8/FITC; anti Qa-2 (irrelevant antibody)—mAb 46/FITC; anti I-Au^{u}-anti I-A^{u}/FITC; anti mouse IgG—goat anti mouse IgG/FITC. Cells used in the experiments were from mouse strains that do not express transferred receptors. To evaluate efficiency of receptor transfer, membrane surface densities were compared to that expressed on cells from expressing mouse strains (Panel A, C (5), Panel B (9)). Results are representative of >20 experiments performed.

To test if protein transfer is cell and cargo specific, various cell types were incubated with avidin/lipid stock and incubated with various biotinylated proteins, H-2D^{b}; I-A^{u}and anti mouse IgG. The cell types examined include resting and activated splenocytes, erythrocytes, p8¹5, and H1 cells.

FIG. 6 demonstrates that the avidin/lipid complex consistently exhibits a high capacity for detergent-free receptor transfer.

Biotinylated, murine MHC class I and MHC class II molecules were transferred with high capacity onto cell membranes of normal, Con A activated splenocytes. Receptor densities of transferred molecules were approximately one log higher as their normal high expression levels (compare histograms 1 and 5 in panel A and histograms 6 and 9 in panel B) and can be estimated to be in the range of millions receptor molecules/cell.

For biotin labeled receptors, interaction of biotin molecule with membrane transferred avidin was essential. Preincubation of avidin binding sites with saturating amounts of free biotin prior to incubation of cells with avidin/lipid stock inhibited receptor transfer completely (histogram 2 in panels A, C and histogram 7 in panel C).

In addition, transfer capacity was proportional to surface area of the membrane. Accordingly, much lower transfer capacity was achieved with smaller erythrocytes as compared to activated splenocytes with the same experimental conditions (compare panel A and C).

Equally good results were obtained with broad range of lipid: avidin molar ratios in labeling reactions ranging from 40:1 to 1:1. High pre-centrifugation of avidin/lipids stock solution (13000 g, 15 min) did not produce pellet or diminish transfer efficiency. Not only avidin/lipids alone but also avidin/lipids complexed with biotinylated reagents can be transferred into membranes as well. When avidin/lipids was preincubated with saturating amounts of biotin/FITC conjugates and unbound fluorochrome labeled biotin was removed by dialysis, FITC-BIO/avidin/lipids complexes transferred into membranes with capacities similarly as demonstrated in two-step incubation protocol. Preformed complexes retain integrity over 1 year period of time when stored at 4° C. without reduced transfer capacity

Additionally, modification by other short lipid 8:0 1,2-dioctanoyl-SN-glycero-3-phosphoethanolamine yielded similar data. In contrast, avidin derivatized with long phospholipids transferred poorly into cell membranes. Avidin/lipids stock solution of long lipids variant developed rapidly precipitates. Incubation of cells with clear supernatant did not result in receptor transfer. Equally negative results were obtained with sonicated or DMSO treated precipitate at wide concentration ranges. Precipitate could be partially solubilized with detergent (n-octylgalactopyranoside). Detergent soluble avidin/lipids transferred poorly into membranes and displayed cytotoxic effect due to the presence of the detergent.

### Example 3

### Detergent-Free Receptor Transfer is Not Toxic for Cells

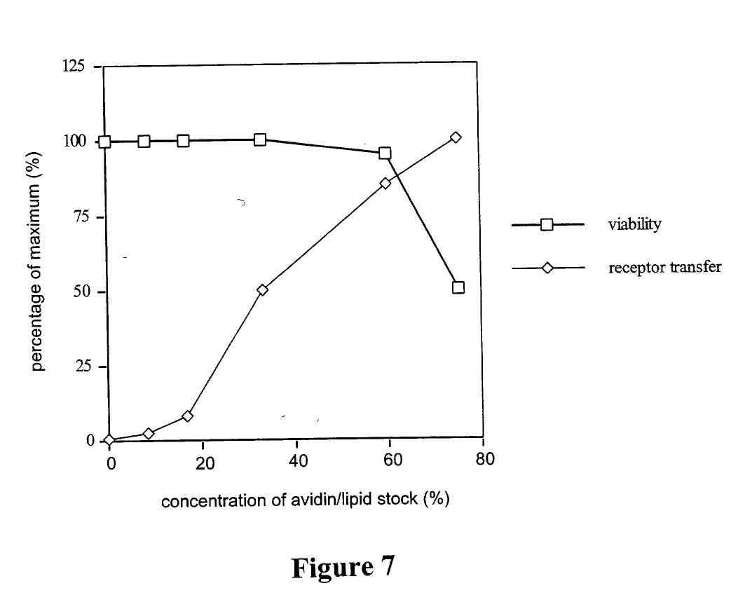

The avidin/lipid transfer complexes were evaluated for toxicity in a range of concentrations. Cell viability was measured using 7 ADD. Typical experimental results with murine tumor cells p815 are shown in FIG. 7. Similar data was obtained with other cells including erythrocytes. Almost no toxic effect has been observed at dilution higher than 3 parts of avidin/lipids with 2 parts of PBS (60% stock). Incubation with undiluted stock solution of avidin/lipids resulted in cell death. Observed toxic effect is more pronounced at higher dilutions of cells. Cell toxicity is probably caused rather by hypotonic incubation conditions at high stock concentrations than by direct toxic effect of avidin/lipids reagent. The range of optimal protein transfer occurs when the avidin/lipid is between 0.3 and 1.5 mg/ml.

### Example 4

### Membrane Density of Transferred Receptors on Viable Cells Decreases with Time

To assess kinetics of expression of transferred receptors, quantities of avidin/lipids or avidin/lipids conjugated to H-2D^{d}BIO were inserted into membranes of viable cells and monitored for the presence of receptor on the surface upon culture by flow cytometry assay.

Activated splenocytes, p815 cells and erythrocytes were isolated and maintained in culture as described in Example 2. On day “0” avidin/lipids (p815 cells) or avidin/lipids+D^{b}BIO (splenocytes or erythrocytes) were transferred on the cell surface as described in Example 2. Cells were cultured subsequently. At indicated time intervals aliquots were withdrawn and expression levels of transferred receptor were monitored on the cell surface of viable cells using flow cytometry.

For p815 cells, viable cells were defined using SSC/FSC and 7 AAD staining profiles. For erythrocytes intact (not lysed) cells were considered viable. Relative expression levels were calculated using Mean Fluorescence Intensity (MFI) values obtained in flow cytometry with receptor specific secondary reagents using the following ratio, MFI of cells with transferred receptors to MFI of untreated cells. Each value was obtained with same flow cytometry settings and using same staining conditions. Background staining of measured and reference cells were identical (as tested with unspecific FITC labeled antibodies). Numeric value of 1 represents no receptor present. For avidin levels (p815) FITC/BIO was used, for D^{b} (splenocytes and erythrocytes) mAb 28-14-8/FITC. This data is representative of two experiments with p815 cells and splenocytes and three experiments with erythrocytes.

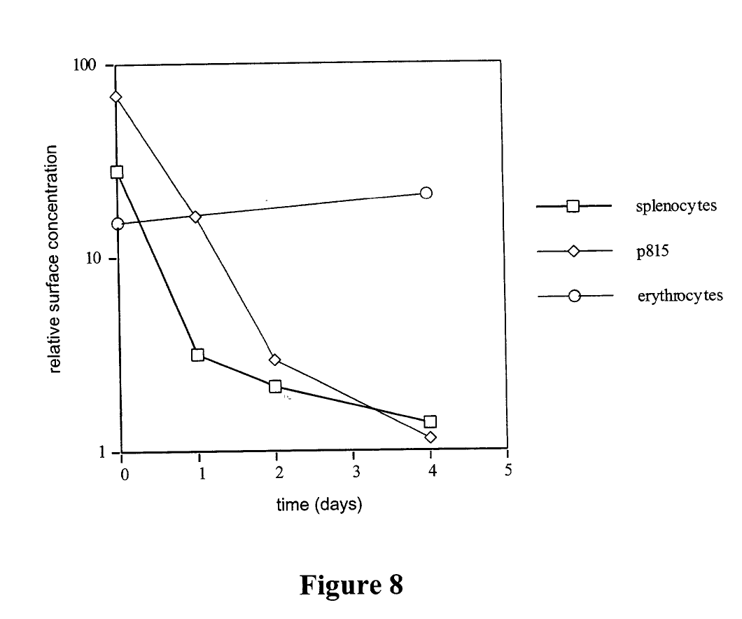

FIG. 8 shows the results the half-life of transferred proteins on splenocytes, p815 cells and erythrocytes. Membrane expression levels decrease rapidly over time in splenocytes and tumor cells due to high membrane metabolism. High level of expression were maintained for 24 hours after transfer and receptors were still expressed up to four days. Levels of expression exceeded physiological levels.

Interestingly, expression was very stable on erythrocytes for prolonged periods of time (more than ten days). This may be explained by the lack of de novo lipid synthesis and membrane traffic in erythrocytes (Percy A. K., Schmell E., Earles B. J. Lennarz W. J. Phospholipid biosynthesis in the membranes of immature and mature red blood cells., Biochemistry, 1973, 12:2464-2461) and the absence of cell divisions in erythrocytes.

Since these results demonstrate that the transferred proteins remain on cell surface for over 24 hours, they will be useful in mediating receptor/ligand interactions leading to cellular responses.

### Example 5

### Transferred Receptors are Functional

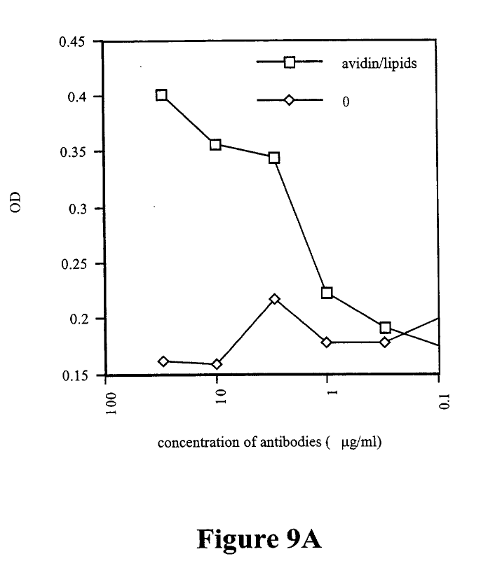

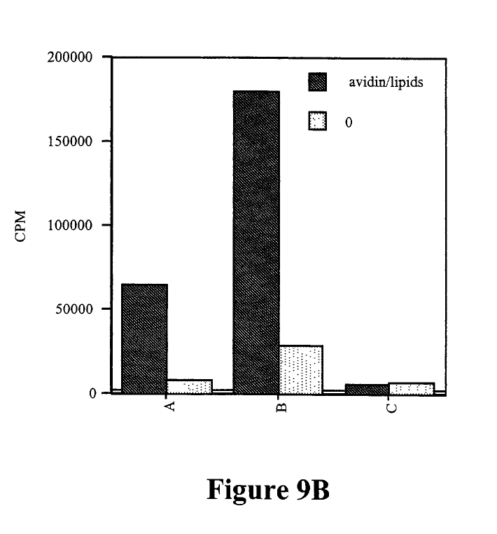

Examples of functional responses are shown in FIGS. 9A and 9B. Upon transfer of stimulatory molecules, responder cells react with adhesion (formation of aggregates), proliferation and secretion of cytokines.

Biotinylated stimulatory antibodies (anti-CD3 and anti-CD28) were transferred on the membrane of freshly isolated, naïve splenocytes as described in Example 2. Cells were cultured subsequently in cRPMI. After 24 hours levels of interferon γ in culture supernatants were measured and plotted against stimulating antibody concentration using ELISA method with commercially available assay kit according to manufacturers protocols. Results are expressed as OD values, which reflect and are proportional to interferon γ levels (FIG. 9A).

Splenocytes were also examined by light microscopy for formation of aggregates. Resting splenocytes cultures in vitro without cytokines do not divide and die within a few days. Aggregation is an indication of cell growth. The splenocytes with transferred stimulatory antibodies were observed to form aggregates in contrast to resting splenocytes that did not form aggregates.

FIG. 9B shows proliferative responses of responder cells incubated for 72 hours with non-stimulating cells (B10PL splenocytes) expressing transferred stimulatory molecules. Activating molecules (antigen specific I-A^{u} class II molecule complexed with stimulatory peptide and second signal delivering molecule anti CD 28) are used in the model of autoimmune disease multiple sclerosis (MS). Responder cells are splenocytes derived from transgenic mice of B10PL background bearing T cell receptor and recognizing I-A^{u} class II molecule complexed with stimulating peptide. B10PL splenocytes were treated with avidin/lipids (dark bars) or without (light bars) (FIG. 9B). Following molecules have been transferred on the surface of B10PL splenocytes: A—anti CD 28BIO and anti CD 3BIO, B—anti CD 28BIO and 1-A^{u}BIO stimulatory MHC/peptide complexes, C—nothing (FIG. 9B). Proliferative responses were measured in standard tritium labeled thymidine incorporation assay. Results in FIG. 9A are representative of four experiments. These results suggest not only that transferred molecules can activate the immune system in an antigen-specific manner, but indicate that the present technology can be potentially useful in the treatment of autoimmune diseases.

### Example 6

### Protein Transfer In Vivo

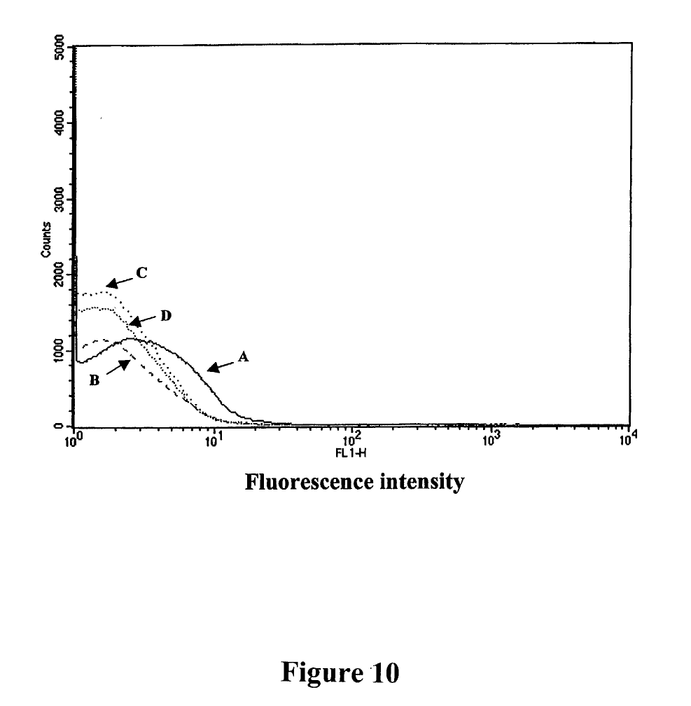

Intrasplenic injections were carried out under sodium pentobarbital (Abbott Laboratories, North Chicago, Ill.) anesthesia (1 mg/mouse in 0.2 ml of PBS by the i.p. route). Spleens were exposed by abdominal incision. 100 μl of avidin/lipids solution (60 μl avidin/lipids stock+40 μl of PBS) or 100 μl of PBS (control mice) were injected directly into organs with 1 ml glass syringes. After time intervals (1 hour, 1 day and two days) mice were sacrificed by cervical dislocation, splenocytes isolated, stained with FITC/BIO and analyzed in flow cytometry. Results from mice treated for 1 hour with avidin/lipid complex are shown in FIG. 10. Avidin/lipid reagent was injected for to collect data for curves A and C and PBS was injected to generate curves B and D. To detect insertions into splenocytes, the isolated cells were stained with FITC/BIO for curves A and B and not stained for curves C and D.

To test if detergent-free receptor transfer can be used for tissue delivery, avidin/lipids have been injected directly into spleens. Results show a small signal after one hour with FITC/BIO stain in comparison to control mice. After one and two days no staining was detected. The small signal may be accounted for by the presence of biotin in the blood which may have blocked avidin binding sites and the suboptimal ratio of avidin/lipids to cell number ratio used in comparison to in vitro condition, i.e. 100 μl avidin/lipids: 2×10⁸ splenocytes vs. 1 ml avidin/lipids: 5×10⁶ in vitro.

The reagent was not toxic to animals and did not cause anaphylactic shock. It was possible to achieve very low level of membrane transfer of avidin/lipids reagent into membranes of splenocytes despite of unfavorable transfer conditions. For this reason it can be claimed that this technology can be applied to deliver receptors into tissues in site specific manner (e.g., tumor site).

I claim:

1. A water soluble complex comprising a soluble receptor attached to a soluble lipid wherein the complex soluble in aqueous media in the absence of detergent or other solubilizing agent.

2. The complex of claim 1 wherein said receptor is selected from the group consisting of peptides, polypeptides, proteins, polysaccharides and glycolipids.

3. The complex of claim 2, wherein the receptor is a protein.

4. The complex of claim 3, wherein the protein is a glycoprotein.

5. The complex of claim 4, wherein the glycoprotein is avidin.

6. The complex of claim 4 wherein the lipid is covalently bound to mannose groups on the glycoprotein.

7. The complex of claim 1 wherein the lipid comprises a fatty acid chain comprising less than 12 carbon atoms.

8. The complex of claim 7 wherein the fatty acid chain comprises less than 9 carbon atoms.

9. The complex of claim 1 wherein said lipid comprises a charged head and a hydrophobic tail.

10. The complex of claim 1 wherein said lipid is selected from the group consisting of phospholipids and cationic lipids.

11. The complex of claim 10 wherein the lipid is a phospholipid having two fatty acid sidechains, wherein each sidechain is less than 12 carbons in length.

12. The complex of claim 11 wherein each sidechain is less than 9 carbons in length.

13. The complex of claim 10 wherein said lipid is a cationic lipid selected from the group consisting of quaternary ammonium salt lipids and lipoamines.

14. The complex of claim 1 wherein the receptor and the lipid are chemically crosslinked.

15. The complex of claim 12 wherein the receptor comprises a thiol group, wherein the thiol group provides attachment to the lipid.

16. The complex of claim 3 further comprising cargo attached to the protein.

17. The complex of claim 16 wherein the cargo is a protein or peptide.

18. The complex of claim 17 wherein the cargo is biotinylated.

19. The complex of claim 3 further comprising a linker having a reactive group for attachment of the linker to the receptor.

20. The complex of claim 19 wherein the linker is selected from the group consisting of polyamines, modified amino acids, and modified nucleotides.

21. The complex of claim 19 wherein the linker is between 10 and 500 angstroms in length.

22. The complex of claim 2 wherein the peptide comprises acidic amino acids and at least one cysteine residue.

23. The complex of claim 22 further comprising cargo attached to the peptide.

24. A composition comprising the complex of claim 1 and a carrier.

25. A method of stimulating an immune response in an animal comprising:
a) producing a soluble complex comprising a soluble receptor attached to a soluble lipid wherein the complex is in the absence of detergent or other solubilizing agent,
b) incubating the complex with cells to allow for insertion of the complex into cell membranes, and
c) injecting cells into an animal to induce an immune response.

26. An immunogenic composition comprising:
a) a mammalian cell capable of stimulating an immune response in a host, said cell having been modified to express an immunogen by insertion of a water soluble complex comprising the immunogen attached to a soluble lipid wherein the complex is inserted into lipid bilayers in the absence of detergent or other solubilizing agent, and
b) an acceptable carrier.
